(19) United States
(12) Patent Application Publication
(10) Pub. No.: US 2022/0259213 A1
(43) Pub. Date: Aug. 18, 2022

(54) PYRROLOPYRAZOLE DERIVATIVES, PREPARATION METHOD THEREOF AND APPLICATION THEREOF IN MEDICINE

(71) Applicants: SHANGHAI MEDICILON INC., Shanghai (CN); MEDICILON PRECLINICAL RESEARCH (SHANGHAI) LLC., Shanghai (CN)

(72) Inventors: Feng REN, Shanghai (CN); Xianlian WANG, Shanghai (CN); Yongmei XU, Shanghai (CN); Chunlin CHEN, Shanghai (CN); Jinna CAI, Shanghai (CN)

(21) Appl. No.: 17/630,117

(22) PCT Filed: Aug. 26, 2019
(86) PCT No.: PCT/CN2019/102526

§ 371 Date: Jan. 25, 2022

(30) Foreign Application Priority Data
Jul. 26, 2019 (CN) 201910682795.1

(51) Int. Cl. C07D 487/04

(52) U.S. Cl. CPC C07D 487/04

(57) ABSTRACT

The present invention relates to pyrrolo-pyrazol derivatives, a preparation method thereof and application thereof in medicine, and particularly to a novel category of pyrrolo-pyrazol derinatives respesented by formula (I), a preparation method thereof and a bio-pharmaceutical use thereof or of pharmaceutical compositions containing the same as therapeutic agents, especially as gastric acid secretion inhibitors and potassium-competitive acid blockers (P-CABs). Substituent groups (Ar and R¹) in formula(I) are defined identically as in description.

### TECHNICAL FIELD

The invention relates to a novel class of pyrrolopyrazole derivatiaves, to a process for their preparation, and to their use as therapeutic agents, especially as inhibitors of gastric acid secretion and as competitive acid blockers (P-CABs) of potassium ion, or pharmaceutical compositions containing them.

### BACKGROUND

Peptic ulcer mainly refers to chronic ulcer that occurs in stomach and duodenum. Although there are regional differences, the incidence of peptic ulcer usually accounts for 10% to 20% of the total population, and is a frequently-occurring disease or a common disease. Ulcer formation is due to various factors, and the digestion of the mucosa by acidic gastric juice is the essential factor in ulcer formation. Therefore, inhibition of gastric acid secretion is becoming the first method for the treatment of peptic ulcer diseases.

Since the first Proton Pump Inhibitors (PPIs) omeprazole was marketed in 1988, several products of PPIs have been marketed globally to date, including lansoprazole, pantoprazole, rabeprazole, and esomeprazole, etc. PPIs have become the first choice drugs for the treatment of gastric acid-related diseases, including peptic ulcer, reflux esophagitis and Zollinger-Ellison Syndrome. The Proton Pump is essentially H⁺/K⁺-adenosine triphosphatase (H⁺/K⁺-ATPase), which specifically pumps protons (H⁺) into the stomach cavity to form a strong acid in the stomach. Proton Pump inhibitors can inhibit the activity of the Proton Pump and thereby regulate the secretion of gastric acid mediated by the Proton Pump.

Potassium-Competitive Acid Blockers (P-CABs) are a novel class of gastric acid blockers that play a role in inhibiting the enzyme activity of H+/K+-ATPase by reversibly binding H⁺/K⁺-ATPase competitively with potassium ions (K⁺). Compared with PPIs, the P-CABs have the characteristics of lipophilicity, alkalescence, stability under acidic (low pH) conditions and the like. At the same time, the P-CABs have the advantages of quick response, easier achievement of acid inhibition effect and the like.

The first new P-CABs drug Voronolazan was marketed in Japan in 2014 for the treatment of gastric acid-related diseases such as peptic ulcer. A series of the structures of potassium ion-competitive acid blocker have also been disclosed. However, there is still a need to develop new compounds with diversified structural types and better medicinal properties.

### SUMMARY

In view of the above-mentioned problems, the object of the present invention is to provide a compound for treating gastric acid-related diseases such as peptic ulcer, which is of a novel structural type and has excellent effects and actions.

In a first aspect, the present invention provides a compound represented by general formula (I) or a pharmaceutically acceptable salt thereof,

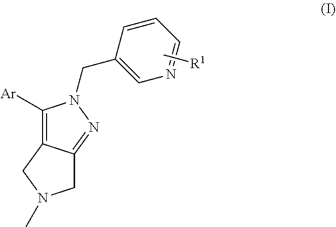

Wherein:

R¹ is selected from hydrogen atom, alkyl or alkoxy;

Ar is selected from a benzene ring or a bicyclic aromatic ring with one or more substituents, and the substituents are each independently selected from hydrogen atom, halogen or alkyl.

Preferably, R¹ is selected from hydrogen atom or C₁₋₃ alkoxy;

Ar is a benzene ring with one or more substituents, the substituents are each independently selected from hydrogen atom or halogen; or Ar is selected from benzimidazole, benzofuran or 2,3-dihydrobenzofuran with one or more substituents, and the substituents are each independently selected from hydrogen atom or halogen.

Preferably, R¹ is selected from hydrogen atom or methoxy;

Ar is a benzene ring with one substituent, and the substituent is selected from hydrogen atom or fluorine atom; or Ar is selected from benzimidazole, benzofuran or 2,3-dihydrobenzofuran.

Preferably, the compound is selected from:

3-(2,3-dihydrobenzofuran-5-methyl)-5-methyl-2-(pyridin-3-ylmethyl)-2,4,5,6-tetrahydropyrrole[3,4-c]pyrazole;

3-(benzofuran-5-yl)-5-methyl -2-(pyridin-3 -ylmethyl)-2,4,5,6-tetrahydropyrrole[3,4-c]pyrazole;

5 -(5-methyl -2-(pyridin-3 -ylmethyl)-2,4,5,6-tetrahydropyrrole[3,4 -c]pyrazol-3-yl)-1H-benzo[d]imidazole;

3 -(2-fluorophenyl)-5-methyl-2-(pyridin-3-ylmethyl)-2,4,5,6-tetrahydropyrrole[3,4-c]pyrazole;

3 -(2-fluorobenzeneyl)-2-((6-methoxypyridin-3-yl)methyl)-5-methyl -2,4,5,6 tetrahydropyrrolo[3,4-c]pyrazole.

In a second aspect, the present invention provides a pharmaceutical composition, comprising the compound represented by general formula (I) or a pharmaceutically acceptable salt thereof, and a pharmaceutically acceptable carrier, excipient or diluent.

In a third aspect, the present invention provides an application of the compound represented by general formula (I) or the pharmaceutically acceptable salt thereof, and the above-mentioned pharmaceutical composition in preparing a gastric acid secretion inhibitor.

In a fourth aspect, the present invention provides an application of the compound represented by general formula (I) or the pharmaceutically acceptable salt thereof, and the above-mentioned pharmaceutical composition in preparing an H⁺/K⁺-adenosine triphosphatase inhibitor.

In a fifth aspect, the present invention provides an application of the compound represented by general formula (I) or the pharmaceutically acceptable salt thereof, and the above-mentioned pharmaceutical composition in preparing a potassium ion competitive acid blocker.

In a sixth aspect, the present invention provides an application of the compound represented by general formula (I) or the pharmaceutically acceptable salt thereof, and the above-mentioned pharmaceutical composition in preparing a medicament for the treatment and/or prevention of peptic ulcer, Zollinger-Ellison Syndrome, gastritis, erosive esophagitis, reflux esophagitis, symptomatic gastroesophageal reflux disease, Barrett's esophagitis, functional dyspepsia, Helicobacter pylori infection, gastric cancer, gastric MALT lymphoma, ulcers caused by non-steroidal anti-inflammatory drugs, or hyperacidity or ulcers caused by post-operative stress; or inhibiting peptic ulcer, acute stress ulcer, hemorrhagic gastritis, or upper gastrointestinal bleeding caused by invasive stress.

### DETAILED DESCRIPTION

The present invention will be further described below through the following embodiments. It should be understood that the following embodiments are only used to illustrate the present invention, not to limit the present invention.

Unless stated to the contrary, the following terms used in the specification and claims have the following meanings.

The term “alkyl” refers to a saturated aliphatic hydrocarbon group, including straight or branched chain groups of 1 to 10 carbon atoms. Preferably, an alkyl groups containing 1 to 5 carbon atoms. More preferably, an alkyl group containing 1 to 3 carbon atoms, such as methyl, ethyl, n-propyl, isopropyl.

The carbon atom content of various hydrocarbon containing moieties is indicated by the prefix designating the minimum and maximum number of carbon atoms for that moiety, i.e., the prefixes Cᵢ₋ⱼ indicate that the number of carbon atoms for that moiety ranges from integers “i” to integers “j” (including i and j). Thus, for example, C₁₋₃ alkyl refers to alkyl groups of 1 to 3 carbon atoms (including 1 and 3).

The term “alkyl” refers to —O-alkyl, where the alkyl is as defined herein.

The term “hydroxy” refers to an —OH group.

The term “halogen” refers to fluorine, chlorine, bromine or iodine.

The term “bicyclic aromatic ring” refers to a bicyclic condensed structure having at least one aromatic ring. The bicyclic aromatic ring preferably contains 0 to 4 heteroatoms selected from O, N and S. Examples of bicyclic aromatic rings are naphthalene ring, benzofuran ring, 2,3-dihydrobenzofuran, benzothiophene ring, indole ring, isoindole ring, benzoxazole ring, benzothiazole ring, indole ring Azole ring, benzimidazole ring, quinoline ring, isoquinoline ring, cinnoline ring, phthalazine ring, quinazoline ring, quinoxaline ring, etc.

Unless otherwise specified, all occurrences of the compounds herein are intended to comprise all possible isomers, such as tautomers, enantiomers, diastereomers, and mixtures thereof

The term “compound of the present invention” refers to a compound represented by the general formula (I). The term also comprises various crystalline forms of the compound of general formula (I), pharmaceutically acceptable salts, hydrates or solvates.

The term “pharmaceutically acceptable salt” refers to salts formed by the compounds of the present invention with acids or bases that are suitable for use as pharmaceutical agents. Pharmaceutically acceptable salts include inorganic salts and organic salts. One preferred class of salts is that formed from the compounds of the present invention and acids. Suitable acids for forming salt include, but are not limited to: inorganic acids such as hydrochloric acid, hydrobromic acid, hydrofluoric acid, sulfuric acid, nitric acid, phosphoric acid, etc., organic acids such as formic acid, acetic acid, propionic acid, oxalic acid, malonic acid, succinic acid, fumaric acid, maleic acid, lactic acid, malic acid, tartaric acid, citric acid, picric acid, methanesulfonic acid, phenylmethanesulfonic acid, benzenesulfonic acid, etc.; and acidic amino acids such as aspartic acid and glutamic acid.

The term “pharmaceutically acceptable carrier” refers to a carrier that can be used in the preparation of pharmaceutical compositions, which are generally safe, non-toxic, not biologically or otherwise undesirable, and comprises carriers that are pharmaceutically acceptable by animals and humans. As used in the specification and claims, a “pharmaceutically acceptable carrier” comprises one or more such carriers.

The terms “comprise”, “contain” or “include” mean that the various ingredients may be used together in a mixture or composition of the present invention. Therefore, the terms “mainly consist of” and “consist of” are encompassed by the term “comprise”.

The term “prevention” refers, for example, to the prevention of development of clinical symptoms of a disease in a mammal that may be exposed to or predisposed to the disease but has not yet experienced or displayed symptoms of the disease.

The term “treatment” may refer to inhibiting a disease, such as preventing or reducing the development of a disease or clinical symptoms thereof, or relieving a disease, such as causing regression of a disease or clinical symptoms thereof.

### Compound of General Formula (I)

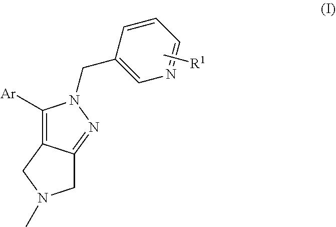

In some embodiments of the present invention, R¹ is selected from hydrogen atom, alkyl, or alkoxy. In a preferred embodiment, R¹ is selected from hydrogen atom or C₁₋₃ alkoxy. In a more preferred embodiment, R¹ is selected from hydrogen atom or methoxy. In a preferred embodiment, R¹ is located at the 6 position on the pyridine ring.

In some embodiments of the present invention, Ar is a benzene ring with one or more substituents. The substituents are each independently selected from hydrogen atom or halogen.

In a preferred embodiment, Ar is a benzene ring with one substituent. The substituent is preferably fluorine atom. In a preferred embodiment, the substituent is located at the 2-position on the benzene ring.

In some embodiments of the present invention, Ar is a bicyclic aromatic ring with one or more substituents. The substituents are each independently selected from hydrogen atom, halogen, or alkyl.

In a preferred embodiment, Ar is selected from benzimidazole, benzofuran or 2,3-dihydrobenzofuran with one or more substituents. The substituents are each independently selected from hydrogen atom or halogen. In a more preferred embodiment, Ar is selected from benzimidazole, benzofuran or 2,3 -dihydrobenzofuran.

In some embodiments of the present invention, the compound of general formula (I) is selected from the compounds shown in Table 1.

TABLE 1

| Compound | | |
|---|---|---|
| number | Compound structure | Compound naming |
| 1 | 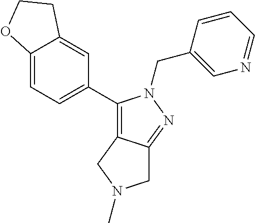 [SMILES: CN1Cc2nn(Cc3cccnc3)c(-c3ccc4c(c3)CCO4)c2C1] | 3-(2,3-dihydrobenzofuran-5- methyl)-5-methyl-2-(pyridin-3- ylmethyl)-2,4,5,6- tetrahydropyrrole[3,4-c]pyrazole |
| 2 | 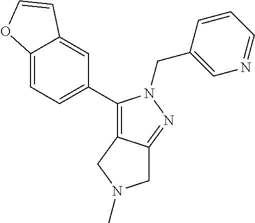 [SMILES: CN1Cc2nn(Cc3cccnc3)c(-c3ccc4occc4c3)c2C1] | 3-(benzofuran-5-yl)-5-methyl-2- (pyridin-3-ylmethyl)-2,4,5,6- tetrahydropyrrole[3,4-c]pyrazole |
| 3 | 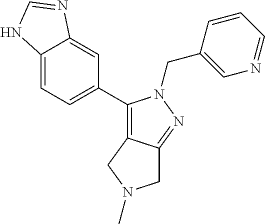 [SMILES: CN1Cc2nn(Cc3cccnc3)c(-c3ccc4[nH]cnc4c3)c2C1] | (5-methyl-2-(pyridin-3-ylmethyl)- 2,4,5,6-tetrahydropyrrole[3,4- c]pyrazol-3-yl)-1H- benzo[d]imidazole |
| 4 | 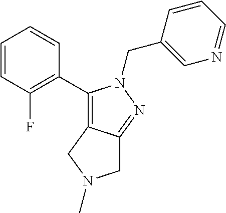 [SMILES: CN1Cc2nn(Cc3cccnc3)c(-c3ccccc3F)c2C1] | (2-fluorophenyl)-5-methyl-2- (pyridin-3-ylmethyl)-2,4,5,6- tetrahydropyrrole[3,4-c]pyrazole |
| 5 | 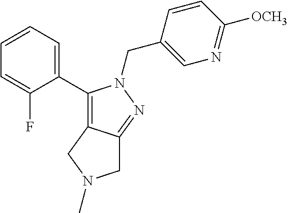 [SMILES: COc1ccc(Cn2nc3c(c2-c2ccccc2F)CN(C)C3)cn1] | 3-(2-fluorobenzeneyl)-2-((6- methoxypyridin-3-yl)methyl)-5- methyl-2,4,5,6- tetrahydropyrrolo[3,4-c]pyrazole |

### Preparation Method of the Compound of General Formula (I)

In some embodiments of the present invention, the compounds of general formula (I) may be prepared using the following general synthetic route:

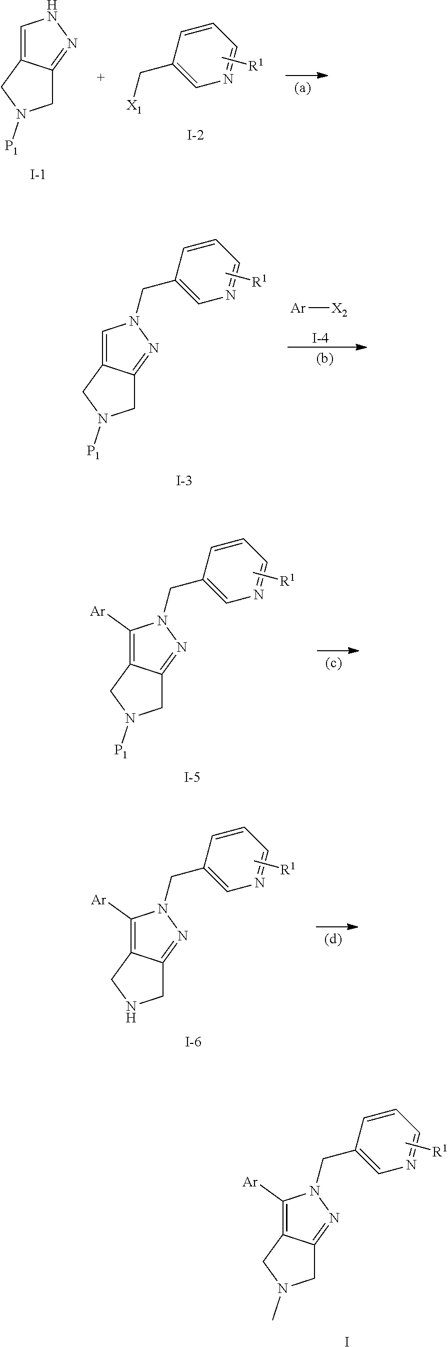

Wherein R¹ and Ar are as defined above.

The P₁ group may be an amino protecting group known in the art, and may be, for example, a C₇₋₁₁ aralkyl group which may be substituted, selected from benzyl, p-methoxyphenylmethyl, o-nitrophenylmethyl, and the like; C₁₋₆ alkylcarbonyl which may be substituted, such as acetyl and trifluoroacetyl; a C₆₋₁₀ arylcarbonyl group which may be substituted, such as benzoyl and the like; and C₁₋₆ alkoxycarbonyl which may be substituted, such as methoxycarbonyl, ethoxycarbonyl, Boc (tert-butoxycarbonyl), Cbz (Benzyloxycarbonyl), Fmoc (fluorenylmethyloxycarbonyl), Teoc (trimethylsilylethoxycarbonyl) and the like; an alkenyloxycarbonyl group such as Alloc (allyloxycarbonyl) and the like; an alkylsulfonyl group such as methylsulfonyl and the like; C₆₋₁₀ arylsulfonyl which may be substituted, such as p-toluenesulfonyl and the like.

The X₁ group may be a leaving group known in the art, and may be selected from, for example, halogen atom such as fluorine atom, chlorine atom, bromine atom, iodine atom, etc.

The X₂ group may be selected from halogen atom such as chlorine atom, bromine atom, iodine atom, etc.

In step (a), the compound of formula I-1 is reacted with the compound of formula I-2 to obtain the compound of formula I-3.

The molar ratio of the compound of formula I-1 to the compound of formula I-2 can be 1: (0.5 to 3.0). The reaction solvent may be acetonitrile, acetone, tetrahydrofuran, dioxane, N,N-dimethylformamide, etc. The reaction of step (a) may be carried out in the presence of a base. The base can be selected from: sodium hydride, cesium carbonate, potassium carbonate, sodium carbonate, potassium hydroxide, sodium hydroxide, and the like. The molar ratio of the compound of formula I-1 to the base can be 1: (1.0 to 6.0). The reaction temperature of step (a) may be appropriately set by those skilled in the art, and may be, for example, 0 to 100° C.

In step (b), the compound of formula I-3 is reacted with the compound of formula I-4 to obtain the compound of formula I-5.

The molar ratio of the compound of formula I-3 to the compound of formula I-4 can be 1: (0.5 to 3.0). The reaction solvent may be acetonitrile, acetone, tetrahydrofuran, dioxane, N,N-dimethylformamide, etc. Step (b) may be carried out in the presence of a palladium catalyst. The palladium catalyst can be selected from: allylpalladium(II) chloride dimer, tris(dibenzylideneacetone)dipalladium, [1,1′-bis(diphenylphosphino)ferrocene] Palladium dichloride, palladium chloride, and the like. Alternatively, the reaction of step (b) may be carried out in the presence of a base. The base may be selected from: potassium acetate, sodium acetate, potassium phosphate, potassium dihydrogen phosphate, potassium bistrimethylsilyl amine, sodium bistrimethylsilyl amine, and the like. The molar ratio of the compound of formula I-3 to the base can be 1: (0.5 to 3.0). The reaction temperature in step (b) may be appropriately set by those skilled in the art, and may be, for example, 40 to 150° C.

In step (c), the P₁ protecting group is removed. The reaction conditions may be those commonly used in the art for deprotecting an amino-protecting group. For example, when P1 is Boc, it can be treated with a protic acid (for example, trifluoroacetic acid) or a Lewis acid.

In step (d), the compound of formula I-6 is subjected to an aminomethylation reaction to obtain the compound of formula I. This step may employ aminomethylation reaction conditions well known in the art. In some embodiments, the compound of formula I-6 is stirred with formaldehyde for a period of time to generate a Schiff base, and then reacted with a reducing agent, such as sodium borohydride acetate, for a period of time to obtain the compound of formula I.

Alternatively, the compound of formula I-3 may be deprotected, aminomethylated and reacted with a compound of formula I-4 to provide a compound of formula I.

### Application of the Compounds of General Formula (I)

The compounds of general formula (I) can be used as inhibitors of gastric acid secretion.

The compounds of general formula (I) can be used as H⁺/K⁺-ATPase inhibitors.

The compounds of general formula (I) can be used as potassium ion competitive acid blockers (P-CABs).

The compounds of general formula (I) can be used for treating and/or preventing peptic ulcer, Zollinger-Ehrlich syndrome, gastritis, erosive esophagitis, reflux esophagitis, symptomatic gastroesophageal reflux disease, Barrett's esophagitis, functional dyspepsia, helicobacter pylori infection, gastric cancer, gastric MALT lymphoma, ulcers caused by non-steroidal anti-inflammatory drugs, or hyperacidity or ulcers caused by post-operative stress; or inhibiting peptic ulcers, acute stress ulcers, Haemorrhagic gastritis or upper gastrointestinal bleeding caused by invasive stress. The aforementioned peptic ulcer includes, but is not limited to, gastric ulcer, duodenal ulcer or anastomotic ulcer. Symptomatic gastroesophageal reflux disease includes, but is not limited to, non-erosive reflux disease or gastroesophageal reflux disease without esophagitis.

### Pharmaceutical Composition

The pharmaceutical composition of the present invention comprises an effective amount of the compound shown in the general formula (I) or tautomer, enantiomer, diastereomer, mixture form thereof, pharmaceutically acceptable salt thereof, and pharmaceutically acceptable carrier or excipient or diluent.

“Effective amount” means the compound of the present invention: (i) treating a particular disease, condition or disorder, (ii) attenuating, ameliorating or eliminating one or more symptoms of a particular disease, condition or disorder, or (iii) preventing or delaying the onset of one or more symptoms of a particular disease, condition, or disorder described herein.

Examples of pharmaceutically acceptable carriers moieties are cellulose and its derivatives (e.g., sodium carboxymethyl cellulose, sodium ethyl cellulose, cellulose acetate, etc.), gelatin, talc, and solid lubricants (e.g., stearic acid, magnesium stearate), calcium sulfate, vegetable oils (e.g., soybean oil, sesame oil, peanut oil, olive oil, etc.), polyols (e.g., propylene glycol, glycerin, mannitol, sorbitol, etc.), emulsifiers (e.g., Tween), wetting agents (e.g., sodium lauryl sulfate), colorants, flavors, stabilizers, antioxidants, preservatives, pyrogen-free water, and the like.

The mode of administration of the compounds or pharmaceutical compositions of the present invention is not particularly limited, and representative modes of administration include (but are not limited to): oral, intratumoral, rectal, parenteral (intravenous, intramuscular, or subcutaneous), and topical administration.

The compounds of the present invention may be administered alone or in combination with other pharmaceutically acceptable compounds.

Another aspect of the present invention relates to a method of inhibiting the secretion of gastric acid, which comprises administering to a patient in need of an effective amount of the compound represented by the general formula (I) or its tautomer, enantiomer, and diastereomer, or mixture thereof, or a pharmaceutically acceptable salt or pharmaceutical composition thereof.

Another aspect of the present invention relates to a method for inhibiting H⁺/K⁺-adenosine triphosphatase (H⁺/K⁺-ATPase) comprising administering to a patient in need of an effective amount of the compound of formula (I) or its tautomers, enantiomers, diastereomers, and mixtures thereof, and pharmaceutically acceptable salts thereof or pharmaceutical compositions thereof.

Hereinafter, the present invention will be further described with the specific examples. It should be understood that the following examples are used to explain this invention and do not mean to limit the scope of this invention. The experimental methods that do not indicate specific conditions in the following examples usually follow the conventional conditions or the conditions recommended by the manufacturer. Unless otherwise stated, percentages and parts are calculated by weight.

The structure of the compound is determined by nuclear magnetic resonance (NMR) or mass spectrometry (MS), and the purity of the compound is determined by liquid high pressure chromatography (HPLC). NMR was measured using a Bruker AVANCE-400 nuclear magnetic resonance apparatus in deuterated dimethyl sulfoxide (DMSO-d6) or deuterated methanol (MeOH-d4) as the solvent and tetramethylsilane (TMS) as the internal standard and chemical shifts in ppm. MS was determined using an Agilent 6120 mass spectrometer. HPLC was measured using an Agilent 1200DAD high pressure liquid chromatograph.

### EXAMPLE 1

### 3 -(2,3-dihydrobenzofuran-5-methyl)-5-methyl -2-(pyridin-3-ylmethyl)-2,4,5,6-tetrahydropyrrole[3,4-c] pyrazole

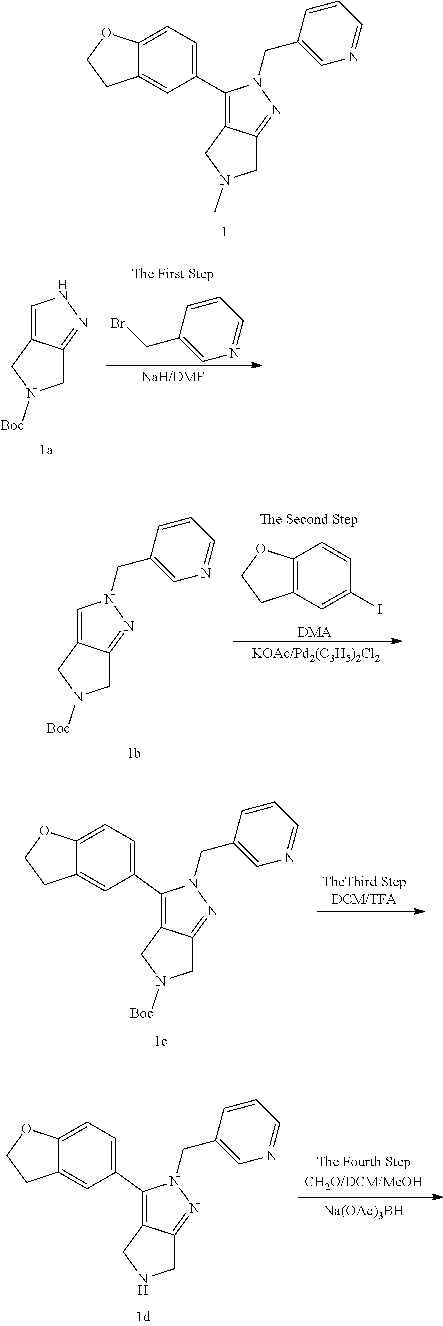

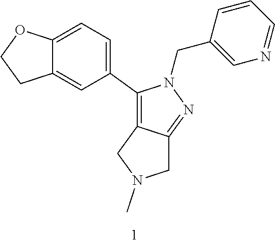 [SMILES: CN1Cc2nn(Cc3cccnc3)c(-c3ccc4c(c3)CCO4)c2C1]

The first step: 2-(pyridin-3-ylmethyl)-2,6-dihydropyrrolo[3,4-c]pyrazole-5(4H)-carboxylic acid tert-butyl ester

The compound, tert-butyl 2,6-dihydropyrrolo [3,4-c] pyrazole-5(4H) -carboxylate 1a (11.25 g, 53.83 mmol) was dissolved in dry DMF (400 mL) and NaH (5.40 g, 60% content, 135 mmol) was added and stirred at room temperature for 1 hour; a solution of 3-bromomethylpyridine hydrobromide (15 g, 59.29 mmol) in DMF (140 mL) was added dropwise and the reaction was continued at room temperature for 1H, water (500 mL) was added to quench the reaction, the reaction was extracted with dichloromethane (500 mL×3), the organic phase was washed with water (200 mL×4), dried, filtered, concentrated and the residue was chromatographed (100% ethyl acetate) to give the crude compound, tert-butyl 2-(pyridin-3-ylmethyl)-2,6-dihydropyrrolo [3,4-c] pyrazole-5 (4H)-carboxylate 1b (14.30g, pale yellow solid), yield: 88.6%. MS M/z (ESI) 301.2[M+1].

The second step: 3-(2,3-Dihydrobenzofuran-5-yl)-2-(pyridin-3-ylmethyl)-2,6-dihydropyrrolo [3,4-c] pyrazole-5(4H)-carboxylic acid tert-butyl ester

The compound, 2-(pyridin-3-ylmethyl)-2,6-dihydropyrrolo[3,4-c] pyrazole-5(4H)-carboxylic acid tert-butyl ester 1b (500 mg, 1.67 mmol), potassium acetate (980 mg, 10.00mmol), allylpalladium (II) chloride dimer (60 mg, 0.17 mmol), 5-iodo-2.3-dihydrobenzofuran (1.02 g, 4.15 mmol) were dissolved in DMA (15 mL) and reacted in a 100 ° C. oil bath for 3 hours under an argon atmosphere. The reaction mixture was cooled to room temperature, poured into water (50 mL), and extracted with ethyl acetate (50 mL×3). The organic phase was washed with saturated aqueous sodium chloride (50 mL×2), dried over anhydrous sodium sulfate, and concentrated. Column chromatography of the residue (100% ethyl acetate) gave the compound 3-(2,3-dihydrobenzofuran-5-yl)-2-(pyridin-3-ylmethyl)-2,6-dihydropyrrolo[3,4-c] pyrazole-5(4H)-carboxylic acid tert-butyl ester 1c (20 mg, yellow solid), yield: 2.8%.

MS m/z (ESI): 419.3[M+1].

The third step: 3-(2,3-dihydrobenzofuran-5-yl)-2-(pyridin-3-ylmethyl)-2,4,5,6-tetrahydropyrrolo[3,4-c] pyrazole

The compound, tert-butyl 3-(2,3-dihydrobenzofuran-5-yl)-2-(pyridin-3-yl methyl)-2,6-dihydropyrrolo[3,4-c] pyrazole-5(4H)-carboxylate 1c (20 mg, 0.048 mmol) was dissolved in dichloromethane (0.9 mL), trifluoroacetic acid (0.3 mL) was added, and the reaction was carried out at room temperature for 1 hour. The reaction was concentrated to give 10 mg of crude compound 3-(2,3-dihydrobenzofuran-5-yl)-2-(pyridin-3-ylmethyl)-2,4,5,6-tetrahydropyrrolo[3,4-c] pyrazole 1d (10 mg, black oil), yield: 65.3%. MS m/z(E.S): 319.2[M +1].

The fourth step: 3-(2,3-dihydrobenzofuran-5-methyl)-5-methyl-2-(pyridin-3-ylmethyl)-2,4,5,6-tetrahydropyrrole [3,4-c] pyrazole

The compound, 3-(2,3-dihydrobenzofuran-5-yl)-2-(pyridin-3-ylmethyl)-2,4,5,6-tetrahydropyrrolo [3,4-c] pyrazole 1d (10 mg, 0.031 mmol) was dissolved in dichloromethane/methanol (2/1, 3 mL), 37% aqueous formaldehyde (21 mg, 0.25 mmol) was added, and the mixture was stirred at room temperature for 0.5 hour, followed by addition of sodium borohydride acetate (53 mg, 0.25 mmol), and reacted at room temperature for 1 hour. The reaction was concentrated and separated by silica gel prep. (dichloromethane/methanol=10/1) to give compound 3-(2,3-dihydrobenzofuran-5-methyl)-5-methyl-2-(pyridin-3-ylmethyl)-2,4,5,6-tetrahydropyrrole [3,4-c] pyrazole 1 (4 mg, yellow solid), yield: 38.8%. MS m/z (ESI): 333.2[M +1]. 1H NMR (400 MHz, CDCl3) δ 8.55(s,1H), 8.34 (s,1H), 7.48 (d, J=7.6Hz,1H), 7.06 (s,1H), 6.99 (d, J=8.3Hz,1H), 6.84 (d, J=8.2Hz,1H), 5.27 (s,2H), 4.65 (t, J=8.8Hz,2H), 4.34 (br,4H), 3.24 (t, J=8.7Hz,2H), 3.03 (s, 3H).

### EXAMPLE 2

### 3-(benzofuran-5-yl)-5-methyl-2-(pyridin-3-ylmethyl)-2,4,5,6-tetrahydropyrrole [3,4-c] pyrazole

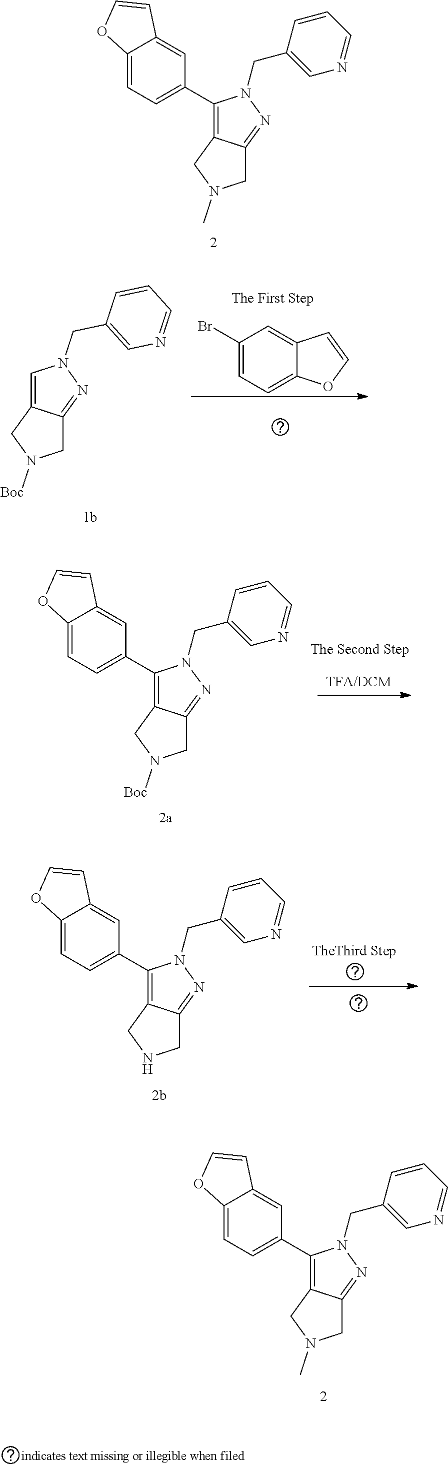

The first step: 3-(benzofuran-5-yl)-2-(pyridin-3-ylmethyl)-2,6-dihydropyrrolo[3,4-c] pyrazole-5(4H)-carboxylic acid tert-butyl ester

The compound, 2-(pyridin-3-ylmethyl)-2,6-dihydropyrrolo[3,4-c] pyrazole-5(4H)-carboxylic acid tert-butyl ester 1b (600 mg, 2.0 mmol), potassium acetate (1.18 g, 12.0 mmol), allylpalladium (II) chloride dimer (73 mg, 0.2 mmol), 5-bromobenzofuran (788 mg, 4.0 mmol), DMA (20 mL) were combined and reacted in a 140° C. oil bath under argon overnight. The reaction mixture was cooled to room temperature, poured into water (60 mL), and extracted with ethyl acetate (30 mL×3). The organic phase was washed with saturated aqueous sodium chloride (50 mL×2), dried over anhydrous sodium sulfate, filtered, and concentrated. HPLC preparation (acetonitrile/water (0.05% NH 3 in gradient rinse)) of the residue afforded the compound 3-(benezofuran- 5-yl)-2-(pyridin-3-ylmethyl)-2,6-dihydropyrrolo[3,4-c] pyrazole-5(4H)-carboxylic acid tert-butyl ester 2a (70 mg, yellow oil), yield: 8.4%. MS M/z (ESI) 417.3[M +1].

The second step: 3-(benzofuran-5-yl)-2-(pyridin-3-ylmethyl)-2,4,5,6-tetrahydropyrrolo[3,4-c] pyrazole

The compound, tert-butyl 3-(benzofuran-5-yl)-2-(pyridin-3-ylmethyl)-2,6-dihydropyrrolo[3,4-c]pyrazole-5(4H)-carboxylate 2a (65 mg, 0.16 mmol) was dissolved in dichloromethane (6 mL), trifluoroacetic acid (2 mL) was added, and the reaction was carried out at room temperature for 2 hours. The reaction was concentrated to give compound 3-(benzofuran-5-yl)-2-(pyridin-3-ylmethyl)-2,4,5,6-tetrahydropyrrolo [3,4-c] pyrazole 2b (40 mg, yellow liquid), yield: 79.1%. MS M/z (ESI) 317.2[M +1].

The third step: 3-(benzofuran-5-yl)-5-methyl -2-(pyridin-3-ylmethyl)-2,4,5,6-tetrahydropyrrolo[3,4-c] pyrazole

The compound, 3-(benzofuran-5-yl)-2-(pyridin-3-ylmethyl)-2,4,5,6-tetrahydropyrrolo[3,4-c] pyrazole 2b (50 mg, 0.16 mmol) was dissolved in dichloromethane/methanol (2/1, 6 mL), an aqueous formaldehyde solution (37%, 103 mg, 1.27 mmol) was added, and the mixture was stirred at room temperature for 1 hour, followed by addition of sodium borohydride acetate (268 mg, 1.27 mmol) and reaction at room temperature for 2 hours. The reaction was concentrated and prepared by HPLC (acetonitrile/water (with 0.05% NH 3) gradient rinse) to give the compound 3-(benzofuran-5-yl)-5-methyl-2-(pyridin-3-ylmethyl)-2,4,5,6-tetrahydropyrrole [3,4-c] pyrazole 2 (16.5 mg, yellow solid), yield: 31.25%. MS M/z (esi) 331.2[M+1]. 1H NMR (400 MHz, CDCl3) δ 8.41 (d, J=4.0 Hz,1H), 8.23 (s,1H), 7.60 (d, J=8.6Hz,1H), 7.47 (d, J=8.Hz,1H), 7.43 (d, J=1.2 Hz,1H), 7.34 (d, J=7.8 Hz,1H), 7.18-7.05 (M,2H), 6.73 (d, J=13.0 Hz,1H), 5.24 (s,2H), 3.87 (s,2H), 3.80 (s,2H), 2.63 (s, 3H).

### EXAMPLE 3

### (5-methyl-2-(pyridin-3-ylmethyl)-2,4,5,6-tetrahydropyrrole[3,4-c]pyrazol-3-yl)-1H -benzo[d]imidazole

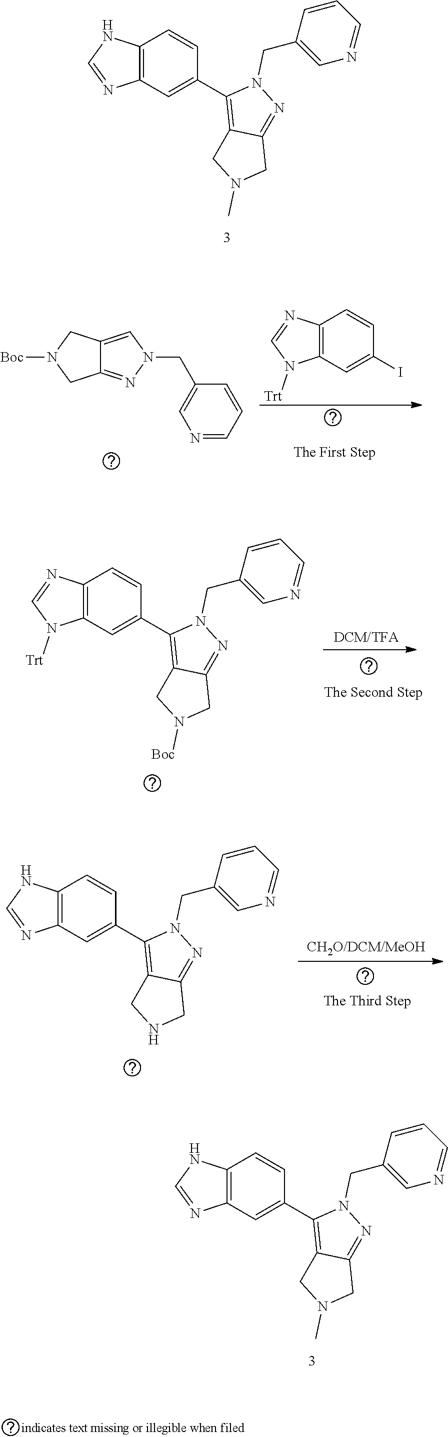

The first step: 2-(pyridin-3-ylmethyl)-3-(1-trityl-1H-benzo[d]imidazol-6-yl)-2,6-dihydropyrrolo[3,4-c] pyrazole-5 (4H)) -carboxylic acid ethyl ester

The compound, 6-iodo-1-trityl-1H-benzo[d] imidazole (1.10 g, 2.28 mmol), potassium acetate (895 mg, 26.54mmol), allylpalladium (II) chloride dimer (55 mg, 0.15 mmol), the compound tert-butyl 2-(pyridin-3-ylmethyl)-2,6-dihydropyrrolo [3,4-c] pyrazole-5(4H)-carboxylate lb (456 mg, 1.52 mmol), DMA (30 mL) were mixed and stirred in a 100° C. oil bath for 2 hours under argon atmosphere. The reaction solution was cooled to room temperature, water (100 mL) was added, extraction was performed with ethyl acetate (50 mL×3), the organic phase was washed with water (10 mL×3), dried over anhydrous sodium sulfate, filtered, concentrated, and the residue was subjected to column chromatography (petroleum ether/ethyl acetate: ⅓—pure ethyl acetate) to give compound 2-(pyridin-3-ylmethyl)-3-(1-trityl-1H-benzo[d] imidazol-6-yl) -2,6-dihydropyrrolo [3,4-c] pyrazole-5 (4H)) -ethyl formate 3a (100 mg, yellow solid), yield: 10%.

The second step: 5-(2-(pyridin-3-ylmethyl)-2,4,5,6-tetrahydropyrrolo [3,4-c] pyrazol-3-yl) -1H-benzo[d] imidazole

The compound, 2-(pyridin-3-ylmethyl)-3-(1-trityl-1H-benzo[d] imidazol-6-yl)-2,6-dihydropyrrolo [3,4-c] pyrazole-5 (4H)) -ethyl formate 3a (100 mg, 0.15 mmol) was added to a mixture of dichloromethane/trifluoroacetic acid (3:1, 8 mL) and reacted at room temperature for 1 hour. The reaction solution was concentrated to give the compound 5-(2-(pyridin-3-ylmethyl)-2,4,5,6-tetrahydropyrrolo[3,4-c]pyrazol-3-yl)-1H-benzo[d] imidazole 3b (40 mg, brown liquid) in 84.3% yield. MS m/z (ESI): 317.2[M+1].

The third step: 5-(5-methyl-2-(pyridin-3-ylmethyl)-2,4,5,6-tetrahydropyrrole[3,4-c] pyrazol-3-yl)-1H-benzo[d] imidazole

The compound, 5-(2-(pyridin-3-ylmethyl)-2,4,5,6-tetrahydropyrrolo [3,4-c] pyrazol-3-yl)-1H-benzo[d] imidazole 3b (320 mg, 1.01 mmol, containing trifluoroacetic acid) was dissolved in a mixed solution of methylene chloride/methanol (2:1, 15 mL), an aqueous formaldehyde solution (37%, 656 mg, 8.10 mmol) was added, and after stirring at room temperature for 30 minutes, sodium borohydride acetate (1.72 g, 8.10 mmol) was added, and the reaction was carried out at room temperature for 2 hours. After the reaction solution was adjusted to weakly alkaline with aqueous ammonia, it was extracted with ethyl acetate (5 mL×3), and the organic phase was washed with a saturated aqueous sodium chloride solution (5 mL×2), dried over anhydrous sodium sulfate, filtered, and concentrated to give compound 5-(5-methyl-2-(pyridin-3-yl methyl)-2,4,5,6-tetrahydropyrrole [3,4-c] pyrazol-3-yl)-1H-benzo [d] imidazole 3 (25 mg, yellow oily substance), yield: 3.0%. MS m/z (ESI): 311.1[M+1]. 1H NMR (400 MHz, MeOD) δ 8.96(s,1H), 8.41(d, J=83.0 Hz,2H), 7.86(d, J=33.9 Hz,2H), 7.70 (d, J=7.8 Hz,1H), 7.50 (d, J=8.1 Hz,2H), 5.54 (s,2H), 4.79-4.42 (m,4H), 3.20 (s, 3H).

### EXAMPLE 4

### (2-fluorophenyl)-5-methyl-2-(pyridin-3-ylmethyl)-2,4 5,6-tetrahydropyrrole [3,4-c] pyrazole

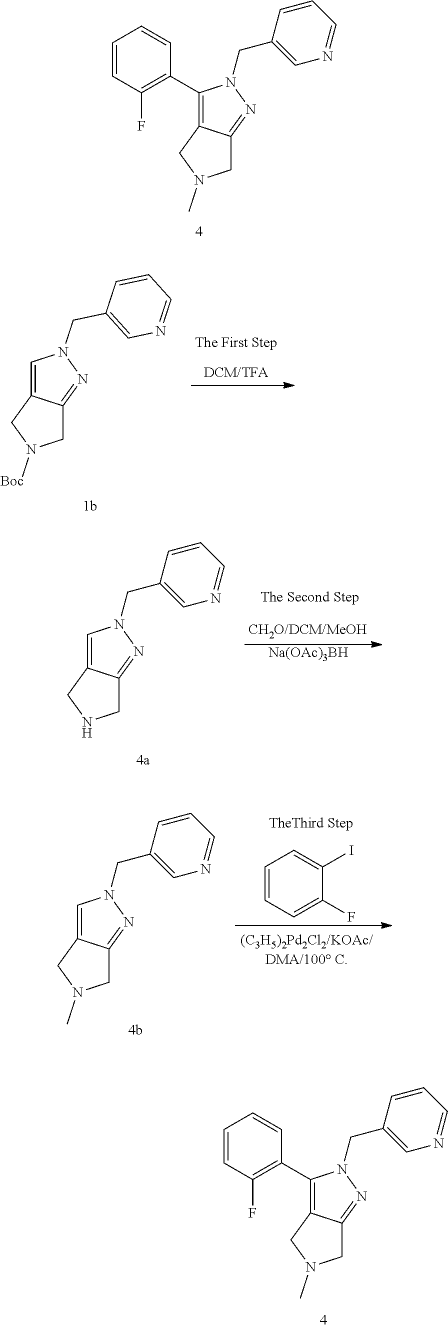

The first step: 2-(pyridin-3-ylmethyl)-2,4,5,6-tetrahydropyrrolo [3,4-c] pyrazole

The compound, tert-butyl 2-(pyridin-3-ylmethyl)-2,6-dihydropyrrolo [3,4-c]pyrazole-5(4H)-carboxylate lb (1.5 g, 5 mmol) was dissolved in dichloromethane (15 mL), and trifluoroacetic acid (5 mL) was added to react at room temperature for 1.5 hours. The reaction was concentrated to give crude compound 2- (pyridin-3-yl methyl)-2,4,5,6-tetrahydropyrrolo [3,4-c] pyrazole 4a (1.0 g, black oil), yield: 100%. MS m/z (ESI): 201.1[M+1].

The second step: 5-methyl-2-(pyridin-3-ylmethyl)-2,4,5,6-tetrahydropyrrolo[3,4-c]pyrazole

Aqueous formaldehyde (37%, 40 mmol, 3.24 g) was added to a solution of compound 2-(pyridin-3-ylmethyl)-2,4,5,6-tetrahydropyrrolo [3,4-c] pyrazole 4a (1.0 g, 5 mmol) in dichloromethane/methanol (2/1, 15 mL), stirred at room temperature for 0.5 h, then sodium borohydride acetate (6.36 g, 30 mmol) was added and reacted at room temperature for 1 h. The reaction was concentrated and column-separated (ethyl acetate) to give 5-methyl-2-(pyridin-3-ylmethyl)-2,4,5,6-tetrahydropyrrolo [3,4-c] pyrazole 4b (1.0 g, yellow oil) in 93.4% yield. MS m/z (ESI): 215.2[M+1].

The third step: 3-(2-fluorophenyl)-5-methyl-2-(pyridin-3-ylmethyl)-2,4,5,6-tetrahydropyrrole [3,4-c] pyrazole

The compound, 5-methyl-2-(pyridin-3-ylmethyl)-2,4,5,6- tetrahydropyrrolo [3,4-c] pyrazole 4b (500 mg, 2.34 mmol), potassium acetate (1.38 g, 14.04 mmol), allylpalladium (II) chloride dimer (85 mg, 0.234 mmol), o-fluoroiodobenzene (1.04 mg, 4.68 mmol), DMA (20 mL) were sequentially added to the reactor and reacted in a 100° C. oil bath under argon overnight. The reaction mixture was cooled to room temperature, poured into water (60 mL), and extracted with ethyl acetate (30 mL×3). The organic phase was washed with saturated aqueous sodium chloride (50 mL×2), dried over anhydrous sodium sulfate, filtered, and concentrated. The residue was prepared by HPLC (acetonitrile/water (with 0.05% NH3 gradient rinse) to give 3-(2-fluorophenyl)-5-methyl-2-(pyridin-3-ylmethyl)-2,4,5,6-tetrahydropyrrolo [3,4-c] pyrazole 4(20.1 mg, yellow oil), yield: 2.8%. MS M/z (esi) 309.2[M+1]. 1H NMR (400 MHz, CDCl3) δ 8.39 (d, J=3.7 Hz,1H), 8.19 (s,1H), 7.31 (dd, J=14.8,7.6 Hz,2H), 7.10 (dd, J=13.3, 6.4 Hz, 4H), 5.17 (s, 2H), 3.73 (d, J=43.3Hz,4H), 2.57 (s, 3H).

### EXAMPLE 5

### 3-(2-fluorobenzeneyl)-2-((6-methoxypyridin-3-yl)methyl)-5-methyl -2,4,5,6-tetrahydropyrrolo[3,4-c]pyrazole

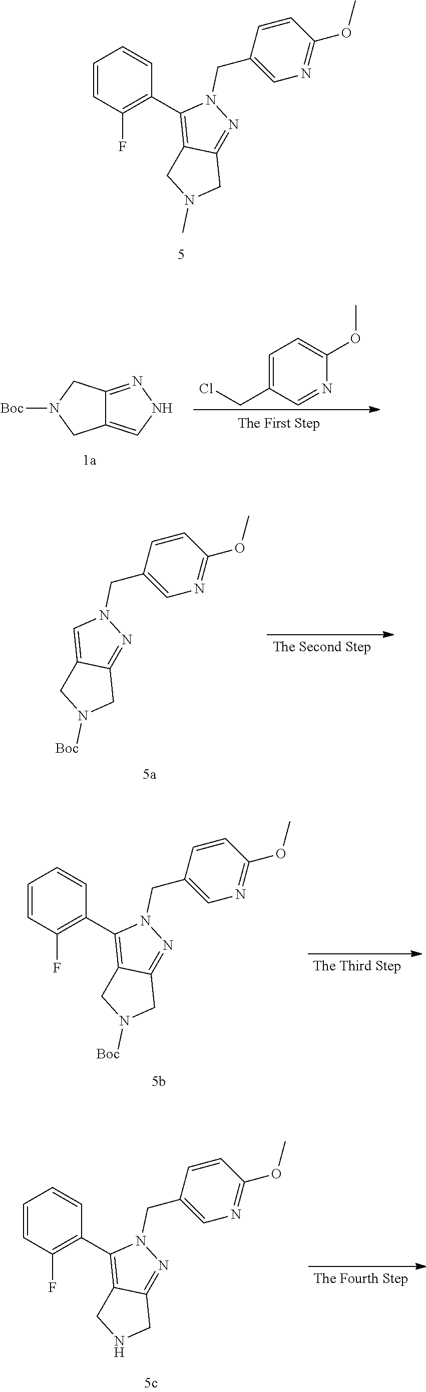

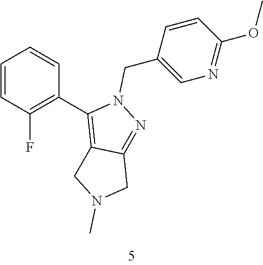 [SMILES: COc1ccc(Cn2nc3c(c2-c2ccccc2F)CN(C)C3)cn1]

The first step: 2((6-Methoxypyridin-3-yl)methyl)-2,6-dihydropyrrolo[3,4-c] pyrazole-5 (4H) -carboxylic acid tert-butyl ester

Taking tert-butyl 2, 6-dihydropyrrolo [3,4-c] pyrazole-5 (4H) -carboxylate 1a (6 g, 28.9 mmol) and sodium hydride (2.3 g, 57.8 mmol) in a round bottom flask, purging the bottle with argon, stirring tetrahydrofuran (80 ml) at room temperature for 1 hour, dropwise adding a tetrahydrofuran (20 ml) solution of 5-(chloromethyl)-2-methoxypyridine (5 g, 31.7 mmol), continuing to react for 1 hour, and heating to 80° C. overnight. The reaction was quenched by slowly adding purified water (20 ml) in ice bath, followed by extraction with ethyl acetate (80 ml), and washing with water (20×2 ml) followed by brine (20 ml). The organic phase was dried over anhydrous sodium sulfate, filtered and concentrated. The crude product was isolated by column chromatography (petroleum ether/ethyl acetate 3/1) to give 2- ((6-methoxypyridin-3-yl) methyl)-2,6-dihydropyrrolo [3,4-c] pyrazole-5(4H)-carboxylic acid tert-butyl ester 5a (6.4 g, yellow solid) in 67% yield. MS m/z (ESI): 331.3[M+1].

The second step: 3-(2-fluorophenyl)-246-methoxypyridin-3-yl)methyl)-2,6-dihydropyrrolo [3,4-c] pyrazole-5 (4H) -carboxylic acid tert-butyl ester

To a round bottom flask were added 2-((6-methoxypyridin-3-yl) methyl) -2,6-dihydropyrrolo [3,4-c] pyrazole-5(4H) -carboxylic acid tert-butyl ester 5a (0.50 g, 1.5 mmol), potassium acetate (0.89 g, 9.1 mmol), allylpalladium (II) chloride dimer (55 mg, 0.15 mmol), N-dimethylacetamide (20 mL), and o-fluoroiodobenzene (0.67 g, 3.0 mmol) in this order. After pumping with oil pump for four times of ventilation (argon), the mixture was put into an oil bath which was raised to 150° C. in advance for reaction for 16 hours. After the reaction was returned to room temperature, the reaction mixture was poured directly into water (40 mL) and extracted with ethyl acetate (20 mL×3). The organic phase was washed with brine (20 ml×2), then dried over anhydrous sodium sulfate, filtered, and concentrated. The crude product was isolated by column chromatography (petroleum ether/ethyl acetate=2/1) to give 3-(2-fluorophenyl)-2-((6-methoxypyridin-3-yl)methyl)-2,6-dihydropyrrolo[3,4-c] pyrazole-5(4H)-carboxylic acid tert-butyl ester 5b (160 mg, pale yellow solid) in 25% yield. MSm/z (ESI): 425.3[M+1].

The third step: 3-(2-fluorophenyl)-2-((6-methoxypyridin-3-yl)methyl)-2,4,5,6-tetrahydropyrrolo [3,4-c] pyrazole

Trifluoroacetic acid (2 mL) was added to a solution of tert-butyl 3-(2-fluorophenyl)-2-((6-methoxypyridin-3-yl)methyl)-2,6-dihydropyrrolo [3,4-c] pyrazole-5(4H)-carboxylate 5b (160 mg, 0.38 mmol) in dichloromethane (6 mL) and reacted at room temperature for 2 hours. After the reaction was completed, 3(2-fluorophenyl)-2-((6-methoxypyridin-3-yl) methyl)-2,4,5,6-tetrahydropyrrolo [3,4-c] pyrazole 5c (110 mg, black oil) was obtained by direct concentration in 89% yield. MS m/z (ESI): 325.2[M+1].

The fourth step: 3-(2-fluorophenyl)-2-((6-methoxypyri din-3-yl)methyl)-5-methyl -2,4,5,6-tetrahydropyrrolo [3,4-c] pyrazole

Aqueous formaldehyde (37%, 214 mg, 2.6 mmol) was added to a solution of 3-(2-fluorophenyl)-2-((6-methoxypyridin-3-yl)methyl)-2,4,5,6-tetrahydropyrrolo [3,4-c] pyrazole (110 mg, 0.33 mmol) in dichloromethane/methanol (6 mL, 2/1) and stirred at room temperature for half an hour. Sodium borohydride acetate (420 mg, 2.0 mmol) was slowly added to the reaction solution, and the reaction was allowed to proceed overnight at room temperature. The reaction mixture was concentrated, dissolved and diluted with dichloromethane (10 ml), washed successively with aqueous ammonia/water (10 ml×2, 1/5) and saturated brine (10 ml), and the organic phase was dried over anhydrous sodium sulfate and concentrated. After concentration the crude product was isolated on a preparative plate (dichloromethane/methanol=10/1) to give 3-(2-fluorophenyl)-2-((6-m ethoxypyri din-3-yl)methyl)-5-methyl -2,4,5,6-tetrahydropyrrolo [3,4-c] pyrazole 5 (80 mg, light yellow solid) in 72% yield. MS m/z (ESI): 339.3[M+1]. 1H NMR (400 MHz, CDCl3): δ 7.84 (d, J=4 Hz, 1H), 7.48-7.46 (m, 1H), 7.37-7.21 (m, 4H), 7.68 (d, J=8Hz, 1H), 5.17 (s, 2H), 4.33 (d, J=36 Hz, 4H), 3.89 (s, 3H), 2.96 (s, 3H).

Test example: determination of compounds' inhibition of H+/K+ATPase enzyme activity

The following experiment is used to determine the inhibitory effect of the compound of the present invention on the H+/K+ATPase enzyme activity.

1. Experimental materials

Plate reader: SpectraMax M5(MD).

Malachite Green (Sigma Aldrich, 213020-25G).

Ammonium molybdate (Sigma Aldrich, 277908-20G).

ATP (Sigma Aldrich, A1852-1VL).

2. Buffer preparation

Enzyme working solution: titrating the enzyme, diluting the enzyme with buffer solution 1, and taking 5 μl of the diluted solution into 50 μl reaction system.

ATP solution: 100 mM ATP was diluted to 5 mM with no K+buffer, and 5 μl of the diluted solution was added to the 50 μl reaction system, that is, the final concentration of ATP was 500 μM.

MLG color development liquid: 0.12% MLG, 7.5% ammonium molybdate, 11% Tween-20 was mixed as 100:25:2, and adding 15 μl of the mixture into each well during detection.

Buffer 1: 5 mM Tris-HCl pH 6.5, 5 mM magnesium chloride (magnesium chloride), 10 μM valinomycin (valinomycin).

Buffer 2: 50 mM Tris-HCl pH 6.5, 5mM magnesium chloride (magnesium chloride), 10 μM valinomycin (valinomycin), 20 mM KCl.

Homogenization buffer: 10 mmol/L Tris-HCl, pH 6.8, 0.25M sucrose (sucrose), 1 mmol/LEDTA.

7.5% Ficoll layering solution: homogenization buffer+7.5% (W/W) (Ficoll 400).

3. Experimental steps

3.1. H⁺/K⁺ ATPase enzyme extraction

(1) The stomach tissue of the rabbit was separated, and the blood was washed with tap water, food residue.

(2) The fundus portion was thoroughly washed with pre-cooled NaCl solution to remove surface mucus.

(3) The stripped mucosa was filled into a sample bag or a 50 ml centrifuge tube, and quickly freezing in a liquid nitrogen tank.

(4) The tissue was removed, minced with surgical scissors, and a pre-cooled homogenization buffer (4 ml/g tissue) was added and homogenized in a tissue homogenizer for 2 to 10 minutes.

(5) After homogenization, if there were larger tissue particles, they could be removed by centrifugation (600 g, 10 min), and then the supernatant was transfered to a clean centrifuge tube. After centrifugation at 20000 g for 30 minutes, then the supernatant was transferred to a clean centrifuge tube at 100000 g for 90 minutes, and the precipitate was collected.

(6) Resuspending the precipitate with homogenization buffer, blowing uniformly, adding 7.5% Ficoll layering solution at equal ratio, centrifuging at 100000 g for 90 minutes, and collecting the precipitate.

(7) Resuspending the precipitate with homogenization buffer, blowing uniformly, and the protein concentration was measured by Bradford. Freezing in tubes at −80° C. for later use.

3.2. H+/K+ATPase activity experiment

(1) Adding 35 μl of reaction buffer to each experimental well, and then adding 35 μl of buffer 1.

(2) Adding 5 μl buffer 1 containing 10% DMSO to the whole enzyme and buffer well.

(3) Adding 5 μl of 10× compound working solution to the compound well and mixing well.

(4) Adding 5 μl of buffer 1 to the buffer well.

(5) Adding 5 μl of 10× enzyme working solution to the remaining wells, mixing and incubating at 37° C. for 30minutes.

(6) Adding 5 μl of 10×ATP working solution to all experimental wells, mixing and incubating at 37° C. for 20 min.

(7) Adding 15 μl MLG chromogenic solution to all experimental wells, and uniformly mixing and incubating at room temperature for 5-30 min.

(8) The reading number of 620 nm was detected by an M5 instrument.

4. Data analysis

The inhibition rate is calculated with the following formula:

Inhibition rate (IC₅₀)=[OD (sample well)−OD (full enzyme well containing potassium chloride)]/[(OD (full enzyme well containing potassium chloride)−(OD (full enzyme well without potassium chloride)]×100%

5. Experimental results

The inhibition rate (IC₅₀) of each example compound is shown in Table 2.

TABLE 2

| Compound number | IC₅₀(μM) |
|---|---|
| Example 1 | 3.55 |
| Example 2 | 1.376 |
| Example 3 | 24 |
| Example 4 | 0.674 |
| Example 5 | 0.3645 |

As can be seen from Table 2, the compounds of the present invention have excellent H+/K+ATPase enzyme inhibitory activity.

1. A compound represented by general formula (I) or a pharmaceutically acceptable salt thereof, 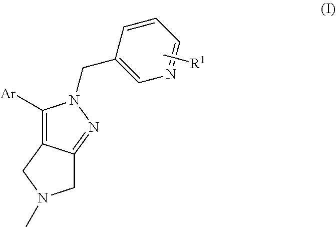
wherein:
R¹ is methoxy;
Ar is selected from a benzene ring with one or more substituents, and the substituents are selected from hydrogen atom, chlorine, bromine or iodine; or Ar is selected from benzimidazole, benzofuran or 2,3-dihydrobenzofuran with one or more substituents, and the substituents are selected from halogen.

2-4. (canceled)

5. A pharmaceutical composition, comprising the compound or a pharmaceutically acceptable salt thereof of claim 1, and a pharmaceutically acceptable carrier, excipient or diluent.

6-8. (canceled)

9. An application of the compound or the pharmaceutically acceptable salt thereof of claim 1 in preparing a medicament for the treatment and/or prevention of peptic ulcer, Zollinger-Ellison Syndrome, gastritis, erosive esophagitis, reflux esophagitis, symptomatic gastroesophageal reflux disease, Barrett's esophagitis, functional dyspepsia, Helicobacter pylori infection, gastric cancer, gastric MALT lymphoma, ulcers caused by non-steroidal anti-inflammatory drugs, or hyperacidity or ulcers caused by post-operative stress; or inhibiting peptic ulcer, acute stress ulcer, hemorrhagic gastritis, or upper gastrointestinal bleeding caused by invasive stress.
